(19) United States
(12) Patent Application Publication
(10) Pub. No.: US 2026/0325251 A1
(43) Pub. Date: Oct. 22, 2026

(54) INTRAORAL APPARATUS AND METHOD OF USE

(71) Applicant: Weiye Lin, Everett, WA (US)

(72) Inventor: Weiye Lin, Everett, WA (US)

(21) Appl. No.: 19/452,442

(22) Filed: Jan. 19, 2026

Related U.S. Application Data
(63) Continuation-in-part of application No. 18/159,416, filed on Jan. 25, 2023, now abandoned.

(51) Int. Cl. A61B 90/00

(52) U.S. Cl. CPC A61B 90/02

(57) ABSTRACT

An intraoral apparatus and methods are disclosed for preventing, reducing, and maintaining the appearance of nasolabial folds through non-invasive intraoral support of facial soft tissues. The apparatus is positioned within an upper oral cavity and includes an upstanding wall having laterally positioned lobes configured to engage inner cheek tissue in regions underlying nasolabial folds. The lobes have increased thickness and smooth tissue-contacting surfaces that cooperate with saliva to generate capillary adhesion, inhibition facial muscle contractions, providing suction-assisted intraoral retention in combination with engagement to the dentition. The lobes are anatomically dimensioned and positioned relative to specific teeth to provide controlled tissue support while minimizing overstretching of surrounding soft tissues. The apparatus may be worn for extended durations, including overnight use, to stabilize facial tissues during resting states and reduce repetitive tissue deformation associated with nasolabial fold formation, without surgical intervention or external facial devices.

### CROSS-REFERENCE TO RELATED APPLICATIONS

This application is a Continuation-in-Part (CIP) of, and claims priority to, a previously filed patent application having U.S. application Ser. No. 18/159,416, filed on Jan. 25, 2023, and titled “Intraoral apparatus and method of use”, the entire disclosure of which is hereby incorporated by reference herein in its entirety.

### FIELD OF THE INVENTION

The present disclosure generally relates to oral and maxillofacial devices, and more particularly to intraoral appliances configured to interact with facial soft tissue for cosmetic and therapeutic purposes. The disclosure further relates to non-invasive systems and methods for influencing facial contours through prolonged intraoral support.

### BACKGROUND

Nasolabial folds are facial creases extending from the lateral aspects of the nose toward the corners of the mouth and are commonly associated with facial expressions such as smiling or laughing. The prominence of these folds tends to increase with age due to factors such as reduction in subcutaneous fat volume, loss of skin elasticity, repetitive facial muscle movement, and gravitational sagging of facial soft tissues. Genetic predisposition, lifestyle factors, and facial anatomy further contribute to the depth and visibility of these folds.

Conventional approaches for addressing nasolabial folds primarily rely on invasive or minimally invasive cosmetic interventions. These include injectable dermal fillers, fat grafting procedures, thread lifting, and surgical techniques such as subcision. While such treatments may provide temporary aesthetic improvement, they typically involve penetration of the skin or underlying tissue and may be associated with adverse effects including bruising, swelling, inflammation, discomfort, and risk of infection. Surgical approaches may further result in scarring, extended recovery periods, and irreversible tissue alteration. Additionally, the outcomes of invasive cosmetic treatments are not permanent and often require repeated interventions to maintain visual results. Repeated procedures increase cumulative cost and may lead to tissue irregularities or an unnatural facial appearance over time. As a result, these approaches may be unsuitable or undesirable for individuals seeking long-term, preventive, or maintenance-based facial aesthetic solutions.

Non-surgical alternatives such as topical formulations, facial exercises, or external devices have been proposed, however, such approaches generally provide limited or inconsistent results due to insufficient mechanical influence on deeper facial structures. Many external devices also rely on visible facial contact, which can interfere with daily activities and user compliance. Accordingly, there remains a need for improved non-invasive, user-friendly, and cost-effective approaches that can be used for long-duration wear and that operate without penetrating the skin, altering tissue surgically, or relying on externally visible facial apparatuses. Such approaches are particularly desirable for preventive use, long-term maintenance of facial appearance, and integration into routine daily or overnight use without disrupting normal activities.

### BRIEF SUMMARY

Devices and methods as described herein relate to preventing or reducing nasolabial folds. These devices, methods, and techniques allow a user to apply an apparatus within a user's mouth on a regular basis for the right amount of time.

According to embodiments, an apparatus for reducing nasolabial folds is described. The apparatus comprises an upstanding wall having a first surface facing a user's lip, the upstanding wall having two leaf shaped lobes positioned apart on either side of the upstanding wall, wherein the lobes extend upward from a top portion of the upstanding wall to the mucobuccal fold region, corresponding to the junction of the labial mucosa and alveolar mucosa, wherein the lobes are of a size and thickness to lift a skin between each corner of a nose and each corner of the lip above from the gum, and wherein the first surface bulges outward at locations of the lobes; and a tray having a second surface facing occlusal surfaces of lower teeth; wherein the upstanding wall and the tray define a third surface having recesses for receiving upper teeth.

According to embodiments, the apparatus further comprises a wire positioned within the upstanding wall, wherein the wire is of substantially the same length as the upstanding wall. According to embodiments, each of the lobes spans between first premolar and first molar of the upper teeth, or between canine and first molar of the upper teeth. According to embodiments, the lobes have a thickness between 0.1 cm to 3 cm. According to embodiments, the lobes have a thickness between 1 cm to 2 cm. According to embodiments, each of the lobes has a height between 1.5 cm to 3 cm from a top portion to a bottom portion of the upstanding wall.

According to embodiments, an apparatus for reducing nasolabial folds is described. The apparatus comprises an upstanding wall having a first surface facing a user's lip, the upstanding wall having two leaf shaped lobes positioned apart on either side of the upstanding wall, wherein the lobes extend upward from a top portion of the upstanding wall to the mucobuccal fold region, corresponding to the junction of the labial mucosa and alveolar mucosa, wherein the lobes are of a size and thickness to lift a skin between each corner of nose and each corner of the lip above from the gum, and wherein the first surface bulges outward at locations of the lobes; and a handle coupled to a front portion of the upstanding wall. According to embodiments, a method of reducing nasolabial folds is described. The method comprises administering an intraoral apparatus configured to be worn daily. The intraoral apparatus comprises an upstanding wall having a first surface facing a user's lip and a second surface facing upper teeth, the upstanding wall having two lobes positioned apart on either side of the upstanding wall, wherein the lobes extend upward from a top portion of the upstanding wall to an area where an upper lip and a gum join, wherein the lobes are of a size and thickness to lift a skin between each corner of nose and each corner of the lip above from the gum, and wherein the first surface bulges outward at locations of the lobes.

According to embodiments, the present invention provides an intraoral apparatus configured for preventing, reducing, and managing the appearance of nasolabial folds through non-invasive, sustained intraoral support of facial soft tissues. The apparatus is adapted to be positioned within an upper oral cavity of a user and is designed to operate entirely intraorally, without reliance on external facial devices or invasive procedures. The invention leverages a combination of anatomical positioning, material selection, surface interaction, and biomechanical retention to influence facial tissue behavior during extended periods of rest, including overnight use.

In one aspect, the intraoral apparatus includes an intraoral support structure having an upstanding wall configured to extend along buccal surfaces of the upper teeth. The upstanding wall includes a top portion oriented toward the upper lip and a bottom portion oriented toward a gingival region, thereby establishing a vertical support profile aligned with maxillary anatomy. The upstanding wall serves as a structural interface between the dentition and facial soft tissues and provides a stable foundation for soft-tissue engagement.

The apparatus further includes two lobes positioned apart on opposing lateral sides of the upstanding wall. Each lobe extends outwardly from the upstanding wall and defines an outwardly bulged tissue-contacting surface having a greater thickness than adjacent portions of the wall. The lobes are configured to engage inner cheek tissue in regions anatomically underlying nasolabial folds, thereby providing localized support and lifting of soft tissue from within the oral cavity.

These two lobes further generate surface adhesion through capillary (meniscus) forces created by a saliva film disposed between the smooth lobe surfaces and the inner cheek. This adhesion produces a suction force that prevents activation of the overlying muscles of facial expression, whether consciously or subconsciously.

Retention of the apparatus is achieved through a dual-mechanism approach. In particular, the apparatus is configured to be retained intraorally by a combination of physical engagement with the upper teeth and surface adhesion between the lobes and buccal mucosa. This surface adhesion is generated through capillary, or meniscus, adhesion formed by a saliva film disposed between smooth lobe surfaces and the inner cheek, thereby providing suction-assisted stabilization that resists displacement caused by facial muscle movement.

In some embodiments, the apparatus further includes a tray configured to cover occlusal surfaces of the upper teeth. The tray cooperates with the upstanding wall to stabilize the apparatus within the upper jaw and distribute retention forces across multiple teeth. In such embodiments, the tray and the upstanding wall define a tooth-receiving surface having recesses configured to receive and conform to the upper teeth, enabling a custom-fit configuration that enhances comfort and positional stability.

In other embodiments, the apparatus may omit an occlusal tray and instead include a smooth inner surface on the upstanding wall configured to contact buccal surfaces of the upper teeth without individual tooth recesses. This configuration enables standardized or over-the-counter implementations while still providing sufficient frictional engagement and suction-assisted retention.

The lobes are dimensioned and positioned to engage facial soft tissues in anatomically relevant regions. Each lobe extends upwardly toward the mucobuccal fold region, corresponding to the junction of the labial mucosa and alveolar mucosa, and extends downwardly below an occlusal plane of the upper teeth. In particular implementations, each lobe spans longitudinally from a mesial region of a first premolar to a distal region of a first molar. In further implementations, each lobe extends downwardly below the occlusal plane by approximately 5 millimeters to 10 millimeters and spans from a mesial surface of a first premolar to approximately a midline of a second molar. These defined spans position the lobes within regions corresponding to maximal nasolabial fold influence while limiting extension into free intraoral space.

Material selection for the lobes is engineered to balance comfort, conformity, and suctional performance. In preferred embodiments, the lobes are formed from a medical-grade elastomeric material having a Shore A hardness in a range of approximately 20 to 60. This range enables soft-on-soft contact with buccal mucosa, allowing intimate surface conformity while maintaining sufficient elastic resistance to sustain capillary adhesion and tissue support during prolonged wear.

The lobe surfaces are further provided with a glassy-smooth surface finish configured to promote formation of a thin saliva film between the lobes and the buccal mucosa. This thin saliva film enhances capillary pressure and generates suction that prevents activation of the overlying muscles of facial expression and minimizes slippage caused by facial muscle movement. The lobe geometry and surface finish are selected to provide sufficient suctional force for stability while minimizing overstretching of surrounding soft tissues and avoiding excessive strain or dysfunction of facial muscles, including the buccinator muscle.

To ensure stability under physiological loading conditions, the lobes collectively define a total tissue-contacting surface area of approximately 4.44 square centimeters. This contact area is selected to resist tangential forces generated by facial muscle movement during use, particularly under nighttime conditions when saliva production is reduced, and capillary adhesion may vary across the contact surface. In some embodiments, the apparatus further includes a handle coupled to the upstanding wall. The handle is configured to facilitate insertion, alignment, and removal of the apparatus from the oral cavity and may be positioned near a midline of the apparatus to assist with proper placement. In additional embodiments, the apparatus includes a bendable reinforcement element positioned within the upstanding wall. The reinforcement element permits manual adjustment of curvature or orientation of the upstanding wall and the lobes, enabling customization of fit, tissue contact, and suctional performance for individual users without compromising structural integrity.

Accordingly, the present invention provides a structurally and functionally integrated intraoral apparatus that combines anatomical support, suction-assisted retention, material engineering, and controlled geometry to non-invasively influence facial soft tissues associated with nasolabial folds during extended intraoral use.

The foregoing summary is illustrative only and is not intended to be in any way limiting. Features from any of the disclosed embodiments can be used in combination with one another, without limitation. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The novel features which are believed to be characteristic of the present invention, as to its structure, organization, use, and method of operation, together with further objectives and advantages thereof, will be better understood from the following drawings in which a presently preferred embodiment of the invention will now be illustrated by way of example. It is expressly understood, however, that the drawings are for the purpose of illustration and description only and are not intended as a definition of the limits of the invention. Embodiments of this invention will now be described by way of example in association with the accompanying drawings in which:

FIG. 1 shows a side perspective view depicting an example intraoral apparatus, according to an exemplary embodiment of the present disclosure.

FIG. 2 shows a back perspective view depicting the example intraoral apparatus of FIG. 1, according to an exemplary embodiment of the present disclosure.

FIG. 3 shows a side perspective view depicting another example intraoral apparatus, according to an exemplary embodiment of the present disclosure.

FIG. 4 shows a back perspective view depicting the example intraoral apparatus of FIG. 3, according to some embodiments.

FIG. 5 shows a top view depicting the example intraoral apparatus of FIG. 1, according to an exemplary embodiment of the present disclosure.

FIG. 6 shows a cross-sectional view along line 6-6 of the example intraoral apparatus of FIG. 5, according to an exemplary embodiment of the present disclosure.

FIG. 7 shows a side view depicting the example intraoral apparatus of FIG. 1, according to an exemplary embodiment of the present disclosure.

Further areas of applicability of the present invention will become apparent from the detailed description provided hereinafter. It should be understood that the detailed description of exemplary embodiments is intended for illustration purposes only and is, therefore, not intended to necessarily limit the scope of the invention.

### DETAILED DESCRIPTION

As used in the specification and claims, the singular forms “a”, “an”, and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the figures may be exaggerated, relative to other elements, to improve the understanding of the present invention. There may be additional components described in the foregoing application that are not depicted on one of the described drawings. In the event such a component is described, but not depicted in a drawing, the absence of such a drawing should not be considered as an omission of such design from the specification.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. Unless stated otherwise, terms such as “first” and “second” are used to arbitrarily distinguish between the elements. Thus, these terms are not necessarily intended to indicate temporal or other prioritization of such elements. While various exemplary embodiments of the disclosed invention have been described below it should be understood that they have been presented for purposes of example only, not limitations. It is not exhaustive and does not limit the invention to the precise form disclosed. Modifications and variations are possible considering the above teachings or may be acquired from practicing of the invention, without departing from the breadth or scope.

In overview, the present disclosure generally relates to intraoral apparatuses and associated methods configured to prevent the formation, slow the progression, and reduce the visible appearance of nasolabial folds. Nasolabial folds are facial creases that extend from lateral regions of the nose toward the corners of the mouth and are commonly accentuated during facial expressions such as smiling, laughing, or speaking. Over time, these folds may become more pronounced due to changes in facial anatomy and soft-tissue support

The development and deepening of nasolabial folds are influenced by multiple physiological factors, including age-related loss of subcutaneous fat, reduction in bone volume, and decreased elasticity of overlying skin. As facial support structures diminish, soft tissues tend to sag and redistribute, increasing the prominence of facial creases even in the absence of active facial expressions. These anatomical changes may occur progressively and are not limited to periods of conscious muscle activity.

In addition to passive structural changes, repetitive contraction of facial muscles contributes to the formation and reinforcement of nasolabial folds. Muscles involved in facial expression, including lip elevators and perioral muscles, insert into the dermal layers of the face. When these muscles repeatedly contract, particularly in regions adjacent to the nose and mouth, localized creasing of the skin may occur and gradually become permanent.

During aging, loss of maxillary and midfacial bone support increases the distance between muscle layers and alters their resting orientation. Such a change in structural alignment can create uneven elevation between adjacent muscle groups, thereby amplifying stress concentrations along the nasolabial region. As a result, facial creases may deepen not only during expressive muscle movements but also while the facial musculature is in a nominal resting state.

The apparatuses disclosed herein are configured to provide an intraoral structural foundation that interacts with facial soft tissues from within the oral cavity. By establishing a controlled and anatomically aligned support plane adjacent to regions of musculodermal insertion, the apparatuses are intended to influence the resting configuration of facial tissues associated with nasolabial fold formation. In particular, the disclosed apparatuses are configured to engage regions associated with muscles of facial expression, including muscles that elevate the upper lip and contribute to nasolabial crease formation. By providing localized intraoral support in proximity to these muscles, the apparatuses may reduce uneven tissue displacement and promote a more uniform resting contour of the facial soft tissues.

The apparatuses are further intended to influence muscle behavior during extended periods of rest, such as during sleep. Facial muscles may remain partially active or assume non-neutral resting positions during sleep, which can contribute to repetitive tissue deformation over time. The disclosed apparatuses are configured to gently modulate such activity by stabilizing adjacent tissues, thereby reducing unintended muscle engagement during resting states.

Through repeated and prolonged use, the apparatuses may promote a more favorable resting condition for facial tissues by reducing localized mechanical stress and allowing muscles involved in facial expression to relax more evenly, in contrast to the progressively uneven underlying skeletal surfaces that develop with aging. Such an effect may support gradual improvement in the appearance of nasolabial folds by limiting further deepening and by facilitating tissue recovery during periods of minimal voluntary muscle activity.

The approaches disclosed herein are non-invasive and are intended to be used without surgical intervention, injectable treatments, or external facial devices. The apparatuses are designed for intraoral placement, thereby avoiding visible facial contact and enabling discreet use during daily activities or overnight wear. In addition, the disclosed apparatuses are intended to provide a low-risk and cost-effective alternative to conventional cosmetic treatments. The designs support repeated or long-term use and may be manufactured as custom-fit or standardized devices, allowing broad applicability for preventive, maintenance, or aesthetic improvement purposes. Accordingly, the present disclosure provides intraoral apparatuses and methods that address nasolabial fold formation by influencing facial soft-tissue support and muscle behavior from within the oral cavity, offering a practical and non-invasive option for managing facial creases associated with aging and repetitive facial expression.

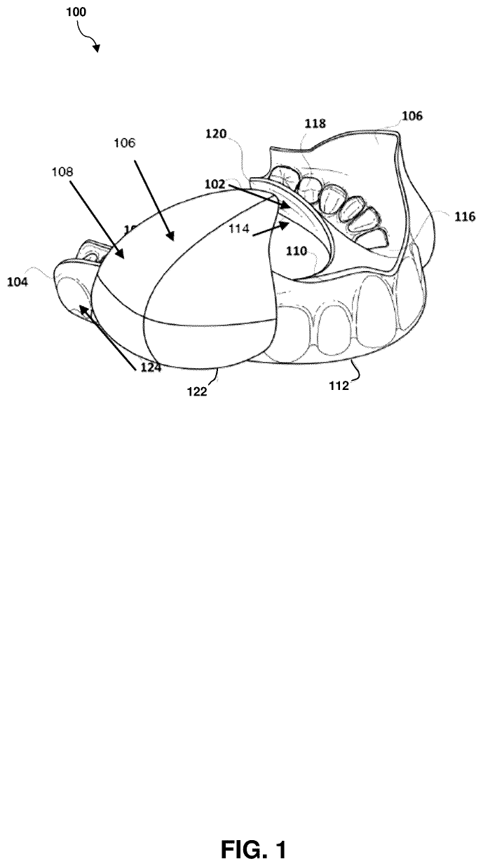

FIG. 1 illustrates a side perspective view of an example intraoral apparatus 100 configured for placement within the upper oral cavity of a user. The intraoral apparatus 100 is structured to be removably seated against the maxillary dentition and adjacent intraoral soft tissues and is configured to interact mechanically with facial soft tissue regions associated with nasolabial fold formation. The apparatus 100 generally includes a tray 102 and an upstanding wall 104 that together define a stable intraoral support structure capable of maintaining positional integrity during extended wear, including during periods of reduced voluntary muscle control such as sleep.

In an embodiment, the tray 102 is configured to cover occlusal surfaces of the user's upper teeth and is shaped to conform to the curvature of the maxillary dental arch. The tray 102 provides a primary anchoring interface with the dentition, distributing retention forces across multiple teeth to avoid localized pressure points. The tray 102 further serves as a foundational structural element from which the upstanding wall 104 extends, thereby transmitting stabilizing forces from the dentition to the facial soft-tissue-engaging regions of the apparatus 100. Extending upwardly from the tray 102 is the upstanding wall 104, which braces against buccal surfaces of the upper teeth. The upstanding wall 104 includes a bottom portion 112 positioned adjacent to the gingival margin and occlusal plane, and a top portion 110 oriented toward the upper lip and buccal mucosa. The distance between the bottom portion 112 and the top portion 110 defines a height of the upstanding wall 104, which is selected to provide sufficient vertical reach to support soft tissue while remaining compatible with intraoral anatomy and comfort constraints.

In an embodiment, the upstanding wall 104 generally has a uniform thickness along regions covering the buccal surfaces of the upper teeth between the top portion 110 and the bottom portion 112. The uniform thickness facilitates even load transfer to the dentition and maintains a consistent profile along the dental arch. In contrast, localized regions of the upstanding wall 104 are intentionally thickened to form lobes 106, which project outwardly from the wall and define functional tissue-contacting structures. The upstanding wall 104 includes a first surface 108 facing outwardly toward the inner cheek and upper lip tissues, and an opposing surface facing inwardly toward the buccal surfaces of the upper teeth. At locations corresponding to the lobes 106, the first surface 108 bulges outward relative to adjacent regions of the upstanding wall 104, thereby creating localized convex contact areas configured to engage and support facial soft tissue from within the oral cavity.

In an embodiment, two leaf shaped lobes 106 are positioned apart from one another on opposing lateral sides of the upstanding wall 104, symmetrically arranged about a midline of the apparatus 100 in typical implementations. Each lobe 106 extends upwardly from the top portion 110 of the upstanding wall 104 toward an area where the mucobuccal fold region, corresponding to the junction of the labial mucosa and alveolar mucosa, including regions adjacent to the maxillary labial frenulum. Such positioning aligns the lobes 106 with anatomical regions underlying nasolabial folds when the apparatus 100 is worn.

In an embodiment, each leaf shaped lobe 106 is configured to engage soft tissue between a corner of the user's nose and a corner of the user's mouth, thereby providing localized lifting and support to tissues that contribute to the visual appearance of nasolabial folds. The lobes 106 apply a controlled outward force that counteracts inward collapse and creasing of the skin caused by loss of underlying structural support, repetitive muscle contraction, and gravitational effects. The height of each lobe 106 is defined as a distance measured from the top portion 110 of the upstanding wall 104 to an uppermost surface of the lobe 106. In various implementations having a height between approximately 1 cm and approximately 2 cm. The thickness of each lobe 106 is defined as the distance between the apex of the surface 108 to the surface of 104 may range from approximately 0.1 cm to approximately 3 cm, with thinnest part at the tip pointing toward the mucobuccal fold region, corresponding to the junction of the labial mucosa and alveolar mucosa. The selected height balances effective tissue engagement with avoidance of excessive upward displacement that could overstretch soft tissue or interfere with normal muscle function. The width of each lobe 106 is defined along a longitudinal direction of the upstanding wall 104 between an upwardly rising curvature and a downwardly descending curvature of the lobe 106. In certain implementations, the width ranges from approximately 3 cm to approximately 5 cm. In preferred configurations, each lobe 106 spans a gum region extending generally from a first premolar 122 to a first molar 124, with the curvature beginning to rise between a canine and the first premolar 122 and descending between the first molar 124 and a second molar.

In an embodiment, the leaf shaped lobes 106 may be symmetrical in height, width, and thickness on opposing sides of the upstanding wall 104. In some implementations, however, the lobes 106 may be asymmetrical to accommodate uneven nasolabial folds or facial asymmetry. Such differential sizing allows targeted support while preserving overall stability and comfort of the apparatus 100. The lobes 106 are further configured to form a suctional interface with the inner cheek surface through surface tension and capillary (meniscus) adhesion. The outwardly bulged first surface 108 at the lobes 106 may be provided with a smooth surface finish that promotes formation of a thin saliva film between the lobe surface and the buccal mucosa. The thin film generates capillary pressure that prevents activation of the overlying muscles of facial expression and enhances adhesion and reduces slippage caused by facial muscle movement.

In preferred implementations, the lobes 106 are formed from a medical-grade elastomeric material, such as silicone, having a Shore A hardness in a range of approximately 20 to 60 (20-60 soft-on-soft ~0.5-1.5 μm High). The material selection enables soft-on-soft conformity with buccal mucosa, allowing intimate surface contact while maintaining sufficient structural resilience to sustain capillary adhesion and tissue support over extended periods of wear.

The suctional interface generated at the lobes 106 acts as a supplemental retention mechanism in addition to mechanical engagement of the tray 102 and upstanding wall 104 with the dentition. The dual-retention approach improves intraoral stability of the apparatus 100 during minor tangential forces generated by facial expressions, while minimizing reliance on excessive bulk or aggressive mechanical clamping that could overstretch soft tissues or strain the buccinator muscle.

In an embodiment, the tray 102 further includes a second surface 114 configured to face occlusal surfaces of lower teeth when the apparatus 100 is worn. The tray 102 and the upstanding wall 104 together define a third surface 116 having a plurality of recesses 118 configured to receive upper teeth. The recesses 118 are custom-fit to the user's dentition, providing precise seating, enhanced comfort, and resistance to lateral and vertical displacement. An inner wall 120 may extend from the tray 102 to further secure the apparatus 100 to the upper jaw. The inner wall 120 cooperates with the recesses 118 to stabilize the apparatus during jaw movement and prolonged wear, distributing forces across multiple teeth and reducing localized stress concentrations.

In some implementations, the upstanding wall 104 may include an internal reinforcement element, such as a wire embedded within the wall, to permit manual adjustment of curvature and fit. Such reinforcement allows controlled shaping of the upstanding wall 104 and lobes 106 to fine-tune tissue contact, suctional performance, and anatomical alignment without compromising structural integrity.

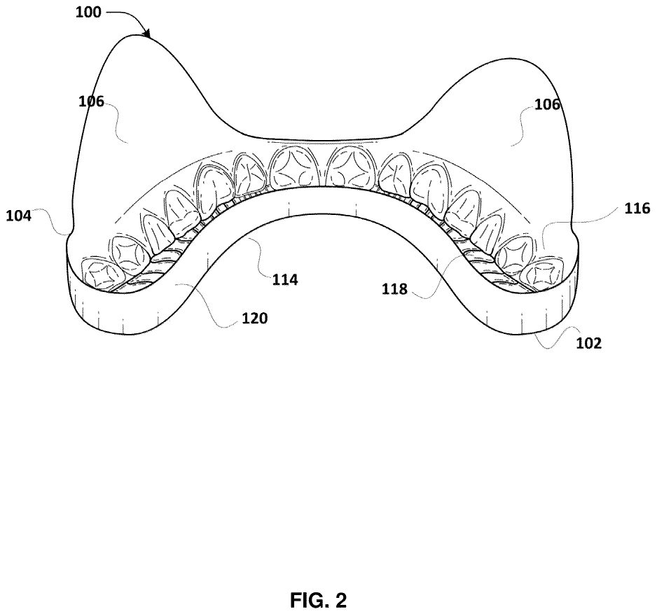

FIG. 2 illustrates a back perspective view of the example intraoral apparatus 100, showing structural features of the apparatus 100 as viewed from a posterior direction when positioned relative to the user's upper jaw. The view emphasizes the overall length, curvature, and material continuity of the tray 102 and the upstanding wall 104 as they extend along the maxillary dental arch. The tray 102 and the upstanding wall 104 are dimensioned to collectively span a sufficient length to cover the user's upper teeth, thereby ensuring stable engagement across multiple teeth rather than relying on localized contact.

As shown in FIG. 2, the tray 102 follows the natural curvature of the upper dental arch and provides a continuous occlusal interface along the posterior-to-anterior direction. The tray 102 is configured to distribute retention forces evenly across the occlusal surfaces of the upper teeth, which contributes to positional stability of the intraoral apparatus 100 during extended wear. The back perspective view further illustrates how the tray 102 integrates seamlessly with the upstanding wall 104, forming a unified structure that resists torsional and lateral displacement.

The upstanding wall 104 extends upwardly from the tray 102 along the buccal side of the upper teeth and maintains a consistent longitudinal profile that conforms to the maxillary arch. From the back perspective, the upstanding wall 104 can be seen to extend continuously along the posterior regions of the dental arch, ensuring that the apparatus 100 remains securely seated even during jaw movement. The height of the upstanding wall 104, defined between the bottom portion 112 and the top portion 110, is selected to provide sufficient reach toward the buccal mucosa while maintaining user comfort.

The lobes 106 are visible from the back perspective as localized thickened regions projecting outwardly from the upstanding wall 104. Each lobe 106 is positioned along a lateral side of the upstanding wall 104 and is oriented to engage soft tissue regions adjacent to the posterior portion of the nasolabial folds. FIG. 2 illustrates how the lobes 106 maintain their relative spacing and curvature along the length of the upstanding wall 104, ensuring symmetrical or intentionally asymmetrical placement depending on the intended configuration. In the illustrated embodiment, the lobes 106 are integrally formed with the upstanding wall 104, resulting in a smooth transition between the wall and the lobes. In alternative embodiments, as further supported by FIG. 2, the lobes 106 may be formed from a material different from that of the tray 102 and the upstanding wall 104. For example, the tray 102 and upstanding wall 104 may be formed from a semi-rigid polymer to provide structural support, while the lobes 106 may be formed from a softer elastomeric material to enhance conformity with buccal mucosa and improve suctional engagement.

The intraoral apparatus 100 may be fabricated as an integrated, unitary structure suitable for prolonged intraoral placement. Materials used for forming the tray 102 and the upstanding wall 104 are selected to balance softness for comfort with sufficient rigidity to maintain the intended geometry of the apparatus 100 under functional loads. Exemplary materials include polymeric materials such as polyethylene terephthalate (PET), polyethylene terephthalate glycol (PETG), high-density polyethylene (HDPE), polypropylene (PP), polyvinyl chloride (PVC), acrylic materials, and other thermoplastic polymers commonly used in dental and medical applications. In some implementations, particularly those intended to enhance suctional adhesion at the lobes 106, the lobes 106 may be formed from a medical-grade silicone material having a Shore A hardness selected to promote soft-on-soft contact with buccal mucosa. Such material selection allows the lobe surfaces to conform closely to the inner cheek, supporting formation of a thin saliva film that generates capillary (meniscus) adhesion, while the tray 102 and upstanding wall 104 maintain sufficient rigidity for mechanical retention against the dentition.

The back perspective view of FIG. 2 further illustrates the smooth surface finish of the upstanding wall 104 and lobes 106, which is selected to minimize irritation and reduce saliva pooling at the interface with intraoral soft tissue. A smoother surface finish contributes to a thinner saliva film at the contact interface, thereby enhancing capillary pressure and improving stability of the apparatus 100 during minor tangential forces generated by facial muscle movement. The intraoral apparatus 100 may be custom-manufactured in a laboratory setting based on an impression, scan, or digital model of the user's dentition. Custom fabrication enables precise shaping of the tray 102, recesses 118 (not shown in FIG. 2), and the curvature of the upstanding wall 104 to match the individual user's dental anatomy. Such customization improves comfort, retention, and alignment of the lobes 106 relative to targeted facial soft-tissue regions. In alternative embodiments, standardized sizes of the intraoral apparatus 100 may be produced, with variations in the overall length, wall height, and lobe geometry to accommodate different mouth sizes. FIG. 2 illustrates a configuration in which the apparatus 100 extends sufficiently into posterior regions of the dental arch to enhance stability while avoiding interference with soft tissues in the retromolar area.

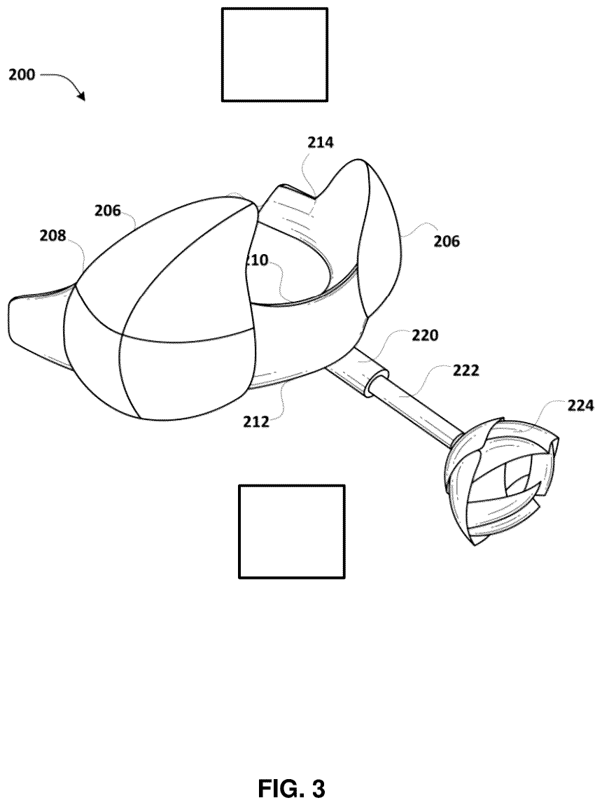

FIG. 3 illustrates a side perspective view of an example intraoral apparatus 200 configured for placement within the upper oral cavity of a user, according to some embodiments. The intraoral apparatus 200 is distinct from the apparatus 100 in that it omits an occlusal tray and instead relies primarily on engagement with the buccal surfaces of the upper teeth and adjacent intraoral soft tissues for retention and functional interaction. Such configuration enables simplified insertion and removal while still providing controlled anatomical support to facial soft tissues associated with nasolabial fold formation.

The intraoral apparatus 200 includes an upstanding wall 204 extending longitudinally along the curvature of the user's maxillary dental arch. The upstanding wall 204 is configured to brace against the buccal surfaces of the upper teeth and to provide a structural interface between the dentition and the facial soft-tissue-engaging portions of the apparatus 200. The upstanding wall 204 includes a bottom portion 212 positioned adjacent to the gingival margin and a top portion 210 oriented toward the upper lip and buccal mucosa, with the distance between the bottom portion 212 and the top portion 210 defining a height of the upstanding wall 204.

In an embodiment, the upstanding wall 204 includes a first surface 208 configured to face outwardly toward the user's cheek and upper lip tissues when the apparatus 200 is worn, and a second surface 214 configured to face inwardly toward the buccal surfaces of the upper teeth. The second surface 214 is substantially smooth and contoured to follow the natural curvature of the dental arch, allowing the upstanding wall 204 to seat comfortably against the teeth without the need for individualized tooth recesses. The configuration supports use of the apparatus 200 as a standardized or over-the-counter device.

In an embodiment, two leaf shaped lobes 206 are formed on the upstanding wall 204 and are positioned apart from one another on opposing lateral sides of the upstanding wall 204. Each lobe 206 extends upwardly from the top portion 210 of the upstanding wall 204 toward the mucobuccal fold region, corresponding to the junction of the labial mucosa and alveolar mucosa, including regions adjacent to the maxillary labial frenulum. The lobes 206 are anatomically positioned to align with intraoral regions underlying the nasolabial folds when the apparatus 200 is placed within the mouth.

In an embodiment, the upstanding wall 204 has a generally uniform thickness along regions covering the buccal surfaces of the upper teeth between the top portion 210 and the bottom portion 212. In contrast, each lobe 206 has a greater localized thickness than the remainder of the upstanding wall 204. The increased thickness produces an outward bulge on the first surface 208 at locations corresponding to the lobes 206, thereby creating convex tissue-contacting regions configured to engage and support facial soft tissue from within the oral cavity. In an embodiment, the lobes 206 are configured to lift and support soft tissue between a corner of the user's nose and a corner of the user's mouth, thereby reducing or minimizing the visual appearance of nasolabial folds. The lobes 206 apply a controlled outward force to counteract inward collapse and creasing of the skin caused by age-related loss of facial support structures, repetitive muscle contraction, and gravitational effects.

In an embodiment, the height of each lobe 206 is defined as a distance measured from the top portion 210 of the upstanding wall 204 to an uppermost surface of the lobe 206. In various implementations, the height of each lobe 206 may range from approximately 1 cm and approximately 2 cm. The thickness of each lobe 106 is defined as the distance between the apex of the surface 208 to the surface of 204 may range from approximately 0.1 cm to approximately 3 cm, with thinnest part at the tip pointing toward the mucobuccal fold region, corresponding to the junction of the labial mucosa and alveolar mucosa. The height is selected to provide sufficient tissue engagement while minimizing overstretching of surrounding soft tissues and avoiding interference with normal facial muscle function. In an embodiment, the width of each lobe 206 is defined along a longitudinal direction of the upstanding wall 204 between an upwardly rising curve and a downwardly descending curve of the lobe 206. In some implementations, the width of each lobe 206 ranges from approximately 3 cm to approximately 5 cm. In preferred configurations, each lobe 206 spans a gum region extending generally from a first premolar to a first molar, with the curvature beginning to rise between a canine and the first premolar and descending between the first molar and a second molar.

The lobes 206 on opposing sides of the upstanding wall 204 may be substantially symmetrical in height, width, and thickness. In some cases, the lobes 206 may be asymmetrical to accommodate uneven nasolabial folds or facial asymmetry in the user. Such asymmetry allows targeted and differential tissue support while maintaining overall stability of the apparatus 200. The lobes 206 are further configured to form a suctional interface with the inner cheek surface through surface tension and capillary (meniscus) adhesion. The outwardly bulged regions of the first surface 208 at the lobes 206 may be provided with a smooth surface finish that promotes formation of a thin saliva film between the lobe surface and the buccal mucosa. The thin saliva film generates capillary pressure that prevents activation of the overlying muscles of facial expression and enhances adhesion and reduces slippage caused by facial muscle movement, and contributes to intraoral retention of the apparatus 200.

In preferred implementations, the lobes 206 are formed from a medical-grade elastomeric material, such as silicone, having a Shore A hardness selected to enable soft-on-soft conformity with buccal mucosa while maintaining sufficient resilience to sustain suctional adhesion and tissue support. The material selection and surface finish are chosen to balance comfort, stability, and effective adhesion without causing irritation at the mucogingival tissue. The suctional interface generated at the lobes 206 serves as a primary or supplemental retention mechanism for the apparatus 200, particularly in the absence of an occlusal tray. The suction-based retention cooperates with frictional engagement between the second surface 214 and the buccal surfaces of the upper teeth to stabilize the apparatus 200 during extended wear and during minor tangential forces generated by facial expressions.

The intraoral apparatus 200 further includes a handle 220 connected to the bottom portion 212 of the upstanding wall 204. The handle 220 extends outwardly from the upstanding wall 204 and is configured to facilitate insertion, alignment, and removal of the apparatus 200. In preferred embodiments, the handle 220 is located near a midline of the apparatus 200 to assist the user in properly centering the device relative to the dental arch. The handle 220 may take various forms, including a flat tab, a substantially cylindrical projection, or other ergonomically shaped extensions. The handle 220 may be integrally formed with the upstanding wall 204 or coupled thereto as a separate component. The geometry of the handle 220 is selected to provide sufficient rigidity for manipulation while minimizing interference with the lips when the apparatus 200 is worn.

In an embodiment, a rod 222 may be connected to the handle 220 and may be configured to extend outwardly from the handle 220. The rod 222 may be rigid or extendable and may serve to increase leverage and improve user control during insertion and removal of the apparatus 200. The rod 222 may be removable or adjustable in length in some implementations. Further, an attachment 224 may be coupled to a distal end of the rod 222. The attachment 224 is configured to provide a grip for the user and may additionally serve an ornamental function. In the embodiment illustrated in FIG. 3, the attachment 224 has a flower-like shape. In other embodiments, the attachment 224 may take the form of a knob, flange, flap, or other graspable geometry without altering the functional purpose of facilitating handling of the apparatus 200.

In some implementations, the upstanding wall 204 and handle 220 may include an internal reinforcement element, such as a wire, to permit controlled bending or shaping of the apparatus 200. Such reinforcement enables customization of curvature and lobe orientation to improve anatomical alignment, suctional performance, and comfort for individual users.

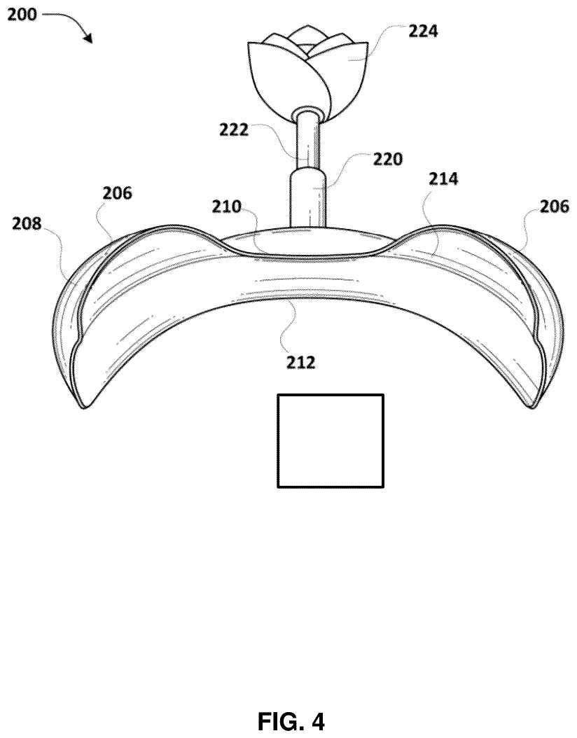

FIG. 4 illustrates a back perspective view of the example intraoral apparatus 200, showing structural and dimensional characteristics of the apparatus 200 as viewed from a posterior direction relative to the user's upper jaw. Such view highlights the overall length, curvature, and continuity of the upstanding wall 204 as it extends along the maxillary dental arch. The upstanding wall 204 is dimensioned to span a sufficient length to cover the user's upper teeth, thereby ensuring stable engagement with the buccal surfaces of multiple teeth rather than relying on localized contact points.

As shown in FIG. 4, the upstanding wall 204 follows the natural posterior-to-anterior curvature of the upper dental arch. The length of the upstanding wall 204 is selected to provide adequate posterior coverage to resist rotational or lateral displacement of the intraoral apparatus 200 during use, while avoiding excessive extension into posterior regions that could interfere with comfort or soft tissue in the retromolar area. FIG. 4 further illustrates that the intraoral apparatus 200 may be provided in a plurality of predefined sizes. In an example embodiment, the apparatus 200 may be available in three sizes that differ from one another in at least one of: the overall length of the upstanding wall 204, the height of the lobes 206 extending from the upstanding wall 204, and the thickness of the lobes 206. These dimensional variations allow the apparatus 200 to accommodate differences in oral anatomy, facial structure, and severity of nasolabial folds among users.

In an embodiment, the selection of an appropriate size of the intraoral apparatus 200 may be facilitated by measuring an anatomical reference dimension of the user's dentition. In one example approach, the distance between the two canines of the upper teeth may be measured and used to determine which size of the upstanding wall 204 and corresponding lobe geometry provides optimal alignment and coverage. The sizing method allows users or clinicians to select a device that positions the lobes 206 in anatomically relevant regions underlying the nasolabial folds.

The back perspective view of FIG. 4 further emphasizes that the second surface 214 of the upstanding wall 204 is substantially smooth and continuous along its length. Unlike the intraoral apparatus 100, the intraoral apparatus 200 does not include recesses for receiving individual upper teeth. Instead, the second surface 214 is contoured to generally match the curvature of the dental arch, allowing the upstanding wall 204 to rest against the buccal surfaces of the upper teeth through broad-area contact. The substantially smooth second surface 214 enables the intraoral apparatus 200 to be used as a standardized or over-the-counter device without the need for individualized tooth impressions. The smooth surface facilitates ease of insertion and removal while still providing sufficient frictional engagement with the buccal surfaces of the upper teeth to contribute to retention and positional stability. The lobes 206 are visible in FIG. 4 as outwardly projecting regions along opposing lateral sides of the upstanding wall 204. Each lobe 206 maintains a defined thickness and curvature relative to the remainder of the upstanding wall 204, and the back perspective view illustrates how the lobes 206 remain integrated with the wall along its length. The positioning and geometry of the lobes 206 are selected to align with intraoral regions underlying nasolabial folds when the apparatus 200 is worn.

In the illustrated embodiments, the intraoral apparatus 200 may be formed as an integrated, unitary structure suitable for prolonged intraoral placement. The material forming the upstanding wall 204 is selected to provide an appropriate balance between softness for user comfort and sufficient rigidity to maintain the intended shape of the apparatus 200 during use. Exemplary materials include elastomeric and polymeric materials such as polysiloxane and polyurethane, which are commonly used in medical and dental applications. In some embodiments, the lobes 206 may be formed from a material different from that of the remainder of the upstanding wall 204. For example, the upstanding wall 204 may be formed from a semi-rigid polymer to provide structural stability and arch conformity, while the lobes 206 may be formed from a softer elastomeric material to enhance conformity with buccal mucosa and improve suctional engagement through surface tension. The material selection and surface characteristics of the lobes 206 are chosen to promote formation of a thin saliva film at the interface between the lobes 206 and the inner cheek surface. The thin film supports capillary (meniscus) adhesion, which cooperates with frictional engagement of the second surface 214 against the buccal surfaces of the upper teeth to retain the intraoral apparatus 200 in a stable position during use.

FIG. 4 further illustrates that the overall geometry of the upstanding wall 204 and lobes 206 avoids excessive bulk or overextension into free intraoral space. Such design minimizes overstretching of surrounding soft tissues and reduces the risk of strain or dysfunction of facial muscles, including the buccinator muscle, while still providing sufficient surface contact area to prevents activation of the overlying muscles of facial expression and maintain stability under minor tangential forces generated by facial expressions. The intraoral apparatus 200 shown in FIG. 4 may be manufactured as a standardized device or may alternatively be custom-fabricated in a laboratory setting based on impressions or digital scans of a user's dentition. Custom fabrication allows refinement of the curvature, length, and lobe geometry of the upstanding wall 204 to improve anatomical alignment, comfort, and suctional performance for individual users.

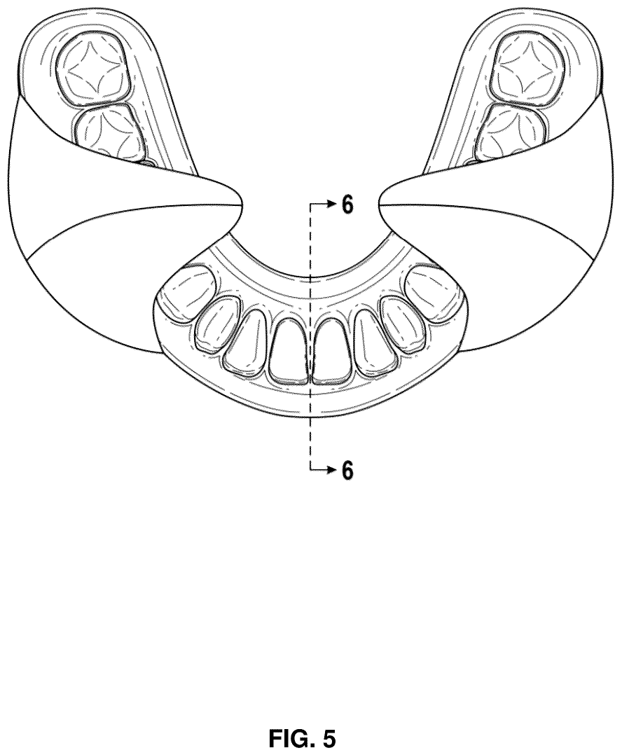

FIG. 5 illustrates a top view of the example intraoral apparatus 100, according to some embodiments, showing the overall planform geometry of the apparatus 100 as viewed along an occlusal direction. From the top view, the tray 102 is visible as extending along the curvature of the maxillary dental arch and defining a continuous occlusal interface configured to receive the upper teeth. The tray 102 is shaped to correspond to the natural arch form of the user, thereby ensuring even distribution of contact forces across multiple teeth and contributing to stable intraoral retention during use.

The top view of FIG. 5 further illustrates the relative positioning of the upstanding wall 104 with respect to the tray 102. The upstanding wall 104 extends along a buccal side of the tray 102 and follows the same arch curvature, thereby maintaining consistent spacing and alignment relative to the upper teeth. The lobes 106 are visible from the top view as laterally positioned regions of increased thickness along the upstanding wall 104, with each lobe 106 occupying a defined longitudinal span corresponding to targeted soft-tissue engagement regions.

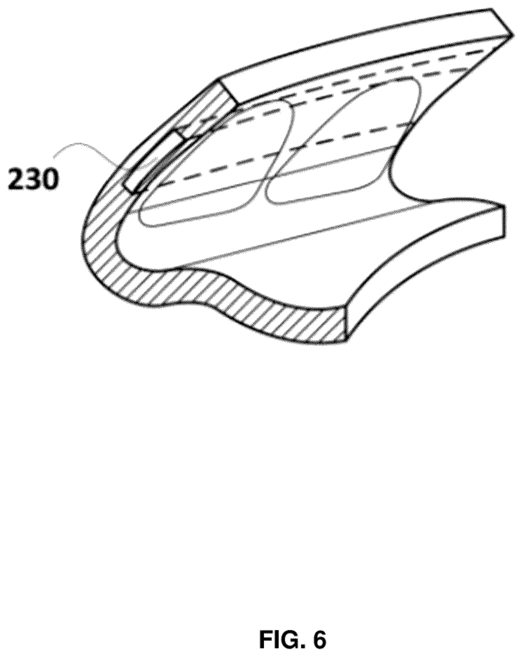

FIG. 6 illustrates a cross-sectional view of the intraoral apparatus 100 taken along line 6-6 of FIG. 5. The cross-sectional view reveals the internal structure of the upstanding wall 104 and its relationship to the tray 102 and surrounding surfaces. In this view, the bottom portion 112 of the upstanding wall 104 is shown extending upward from the tray 102, while the top portion 110 is oriented toward the buccal mucosa. The cross-section further illustrates the outward bulge of the first surface 108 at the location of the lobes 106 relative to the more uniform thickness of the remainder of the upstanding wall 104. As shown in FIG. 6, the tray 102 and the upstanding wall 104 together define the third surface 116, which includes recesses 118 configured to receive and conform to the user's upper teeth. The recesses 118 are shaped to match the contours of the teeth, thereby providing a custom-fit interface that resists vertical and lateral displacement of the intraoral apparatus 100 during jaw movement. The inner wall 120 may also be visible in cross-section, extending upwardly from the tray 102 to further secure the apparatus 100 to the upper jaw.

FIG. 6 further illustrates that the upstanding wall 104 may include a wire 230 positioned within the wall. The wire 230 extends longitudinally along at least a substantial portion of the upstanding wall 104 and is embedded within the wall material. The wire 230 may be formed from a solid yet bendable material, such as metal, elastic material, or fiberglass, and is configured to permit controlled manual shaping of the upstanding wall 104 and lobes 106 to improve anatomical fit and tissue contact. The presence of the wire 230 allows a user or clinician to adjust the curvature, orientation, and contact pressure of the upstanding wall 104 and lobes 106 without compromising the overall structural integrity of the apparatus 100. Such adjustability enables fine-tuning of the suctional interface formed between the lobes 106 and the buccal mucosa, as well as optimization of alignment with the dentition and facial soft tissues. Although not shown in FIGS. 5 and 6, it is understood that the intraoral apparatus 200 may similarly include a wire positioned within the upstanding wall 204. Such a wire may perform an analogous function by enabling adjustment of curvature and lobe positioning in the tray-less embodiment. In addition, the handle 220 of the intraoral apparatus 200 may also include a wire inserted therein to provide additional rigidity or controlled bendability for manipulation during insertion and removal.

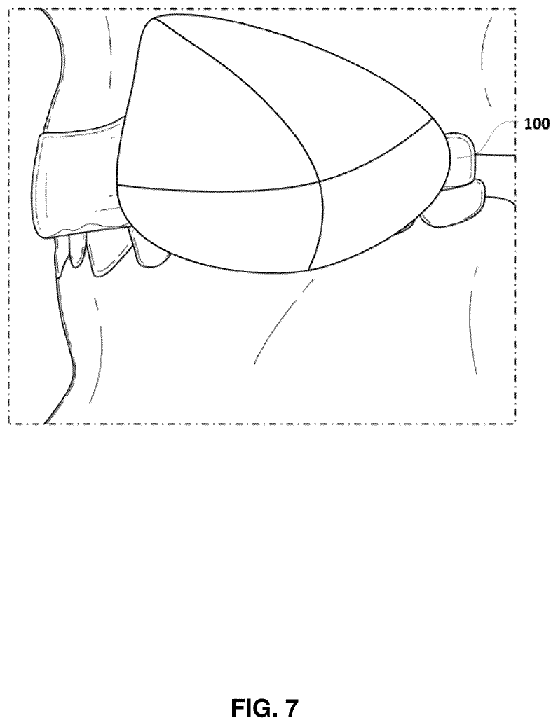

FIG. 7 illustrates a side view of the example intraoral apparatus 100 positioned within the user's mouth, according to some embodiments. In this view, the tray 102 is shown seated against the occlusal surfaces of the upper teeth, while the upstanding wall 104 extends upwardly along the buccal surfaces of the teeth toward the upper lip and inner cheek. The apparatus 100 is positioned entirely within the oral cavity and does not require external facial contact.

As illustrated in FIG. 7, the lobes 106 extend upward from the top portion 110 of the upstanding wall 104 and engage the inner cheek and upper lip tissues in regions underlying the nasolabial folds. The outward bulge of the first surface 108 at the lobes 106 provides localized support and lifting of the facial soft tissues, thereby influencing the contour of the nasolabial region from within the oral cavity. FIG. 7 further illustrates how the intraoral apparatus 100 remains stably positioned within the upper jaw during use. Mechanical engagement of the tray 102 and recesses 118 with the dentition cooperates with suctional adhesion at the lobes 106 to resist displacement caused by facial muscle movement or gravity. The combined retention mechanism enables prolonged wear, including overnight use, while maintaining comfort and minimizing irritation. The positioning shown in FIG. 7 demonstrates that the intraoral apparatus 100 supports facial soft tissues without overfilling available intraoral free space. The geometry of the upstanding wall 104 and lobes 106 is selected to provide sufficient contact area for inhibition of facial muscle contraction, device stability, and tissue support while avoiding excessive stretching of surrounding soft tissues or undue strain on facial muscles, including the buccinator muscle.

### Example Embodiments and Methods of Use

In some embodiments, the present disclosure provides methods for preventing the formation of nasolabial folds, slowing the progression of existing nasolabial folds, and reducing the visual prominence of nasolabial folds through repeated, non-invasive intraoral use of the disclosed apparatuses. The methods are configured for long-duration, low-risk use and are intended to influence facial soft-tissue positioning and muscle behavior during extended periods of rest.

In one example method, an intraoral apparatus, such as the intraoral apparatus 100 or the intraoral apparatus 200, is placed inside a user's mouth and positioned within the upper jaw. The apparatus is seated such that it is secured against the user's upper teeth, with the upstanding wall and associated lobes positioned to engage buccal and labial soft tissues adjacent to regions underlying the nasolabial folds. Proper positioning may be achieved through mechanical engagement with the dentition, suctional adhesion with the inner cheek surface, or a combination thereof, as previously described. Once positioned, the intraoral apparatus is worn for an extended duration on a regular basis. In some embodiments, the apparatus is worn for approximately 6 to 8 hours per day. Such duration is particularly suitable for overnight use during sleep, when voluntary facial muscle activity is reduced and the facial tissues remain in a relatively static state for prolonged periods. Through repeated daily use over time, the apparatus provides sustained intraoral support to facial soft tissues associated with nasolabial fold formation. By maintaining a controlled and anatomically aligned support interface during resting states, and inhibition of facial muscle contraction the apparatus may reduce repetitive tissue collapse and creasing that otherwise occur due to gravity, muscle tone, and age-related loss of structural support.

In some embodiments, users may begin to observe visible improvements in the appearance of nasolabial folds after several weeks or months of consistent use. For individuals with mild nasolabial folds or for preventive use, noticeable changes may occur after approximately three months of regular wear. In cases involving more pronounced or deeper nasolabial folds, longer periods of use, such as six months to one year, may be required to achieve observable results.

The effectiveness of the disclosed methods may vary depending on factors such as age, skin elasticity, facial anatomy, and consistency of use. In some embodiments, users who begin use at a younger age, for example before approximately 25 years of age, may experience more pronounced preventive benefits due to greater tissue elasticity and reduced cumulative structural degradation.

The intraoral apparatus used in the method may be obtained in different forms depending on user preference and clinical requirements. In some embodiments, the intraoral apparatus 100 may be custom-manufactured in a laboratory setting based on impressions or digital scans of the user's dentition to achieve a personalized fit. In other embodiments, the intraoral apparatus 200 may be provided as a standardized or over-the-counter device available through retail channels such as pharmacies.

In additional embodiments, the disclosed methods may be used to maintain or prolong the visual results achieved through other cosmetic treatments, such as injectable filler procedures. Following a filler treatment, the intraoral apparatus may be used as a maintenance device to support facial soft tissues during rest and reduce mechanical stresses that could otherwise accelerate degradation or migration of filler material. In such maintenance applications, the intraoral apparatus may be self-administered by the user, typically during overnight wear for approximately 6 to 8 hours per day. The apparatus may be used on a daily or periodic basis, depending on the desired maintenance regimen and clinical recommendations. By providing non-invasive intraoral support during resting states, the disclosed methods may reduce repeated mechanical deformation of facial soft tissues and may assist in preserving a more uniform resting contour of the nasolabial region. The approach offers a complementary or alternative strategy to invasive or minimally invasive cosmetic interventions. Accordingly, the example embodiments described herein provide methods that leverage prolonged, controlled intraoral tissue engagement to prevent, reduce, or maintain the appearance of nasolabial folds, while avoiding surgical procedures, external facial devices, or repeated injectable treatments, and while enabling convenient and discreet use by the user in everyday or overnight settings.

In summary, the present invention relates to intraoral apparatuses and associated methods for preventing the formation of nasolabial folds, slowing the deepening of nasolabial folds, reducing the visible appearance of nasolabial folds, and maintaining post-treatment facial appearance through non-invasive intraoral support and stabilization of facial soft tissues. The invention is configured to operate entirely within the oral cavity and to provide a low-risk, user-administered approach suitable for long-duration wear, including overnight use, without requiring invasive procedures, external facial devices, or repeated clinical interventions.

The present invention is based on the recognition that nasolabial folds arise from a combination of age-related anatomical changes and repetitive muscle-driven deformation. Contributing factors include reduction in cheek fat volume, sagging of loose skin, loss of facial bone structure, and changes in relative elevation and spacing between facial muscle layers. These factors can cause creasing along musculodermal insertion regions and can deepen lines not only during active facial expressions but also during resting states, including during sleep. The invention therefore addresses both passive structural collapse of soft tissue and expression-related tissue deformation by establishing controlled intraoral support adjacent to intraoral tissues corresponding to the nasolabial region.

In some implementations, the apparatus includes an intraoral support structure positioned within the upper oral cavity and configured to be secured against the user's upper teeth, such that functional elements are placed in proximity to the mucobuccal fold region, corresponding to the junction of the labial mucosa and alveolar mucosa, including regions adjacent to the maxillary labial frenulum. The apparatus includes laterally positioned lobes configured to apply controlled outward lifting and support to soft tissue between the corner of the nose and the corner of the mouth, thereby reducing or minimizing the appearance of nasolabial folds through sustained intraoral support during prolonged wear. The lobes are anatomically sized and positioned with both an upper lobe portion and a lower lobe portion, each having a defined dental span relative to specific teeth. The upper lobe extends from the mesial of the first premolar to the distal of the first molar. The lower lobe extends approximately 5 mm to 1 cm below the occlusal plane and spans from the mesial surface of the first premolar to the midline of the second molar. The combined span corresponds anatomically to the mesial of the lower first premolar through the midline of the lower second molar, and provides a controlled engagement zone aligned to soft-tissue regions underlying nasolabial folds while limiting extension into free intraoral space.

The present invention further provides a suctional retention interface between the lobe surfaces and the inner cheek, in addition to conventional physical attachment to the dentition. The suctional retention is produced through capillary (meniscus) adhesion via a saliva film disposed between a smooth lobe surface and the buccal mucosa. Thinner saliva films generate stronger suction effects due to increased capillary pressure, and the invention therefore specifies material selection and surface finish parameters that promote thin-film formation while maintaining comfort and safety near sensitive intraoral regions around the mucogingival tissue.

Material selection may be based on contact mechanics and saliva film behavior. For a polished metal surface providing hard-on-soft contact, the average saliva film thickness is approximately 0.2-1.0 micrometers, which can produce high capillary pressure; however, such metal materials are typically too rigid for safe and comfortable use near the mucogingival junction. For medical-grade silicone providing soft-on-soft contact, the average saliva film thickness is approximately 0.5-1.5 micrometers, which yields high adhesion due to good conformity and pressure distribution. For acrylic denture resin providing semi-rigid-on-soft contact, the average saliva film thickness is approximately 1.0-3.0 micrometers, which yields moderate suction performance dependent on fit. The invention therefore prioritizes medical-grade silicone as a primary material for the suctional lobe surfaces, while allowing alternative materials and combinations where appropriate.

The present invention further specifies material stiffness using durometer scales to characterize functional behavior. Medical-grade silicone is specified in a range of Shore A 20-60, providing soft-on-soft contact with approximately 0.5-1.5 micrometers saliva film thickness and high adhesion due to conformity and pressure distribution. Acrylic (PMMA) is characterized in a range of Shore D 75-90, providing semi-rigid-on-soft contact with approximately 1-3 micrometers saliva film thickness and moderate adhesion that depends on fit. Metal is characterized in a range of Shore D approximately 85-95, providing hard-on-soft contact with approximately 0.2-1.0 micrometers saliva film thickness and high adhesion in central areas with lower adhesion at edges. These values are used to justify why a softer elastomeric material is preferred to achieve clinically acceptable comfort while still producing effective capillary adhesion.

Surface finish is treated as a functional parameter for achieving meniscus adhesion. A smoother surface retains less fluid, producing a thinner saliva film and thus higher capillary pressure, which enhances adhesion and that hold facial muscle in position and prevent movement and at the same time minimizes slippage caused by such muscle movement. The invention therefore specifies a glassy-smooth surface finish for the intraoral suction surfaces, particularly when formed of medical-grade silicone, to achieve a stable suctional interface while maintaining flexibility and comfort. Although metal surfaces can provide the greatest capillary pressure due to minimal surface texture and thinner films, the invention recognizes that rigid metal is unsuitable for the application due to comfort limitations and potential irritation to mucogingival tissue, and therefore selects silicone with a glassy-smooth finish as a preferred implementation.

The present invention further provides quantitative engineering for contact area and retention under realistic physiological forces. To resist a representative tangential force of approximately 2 newtons applied tangent to the device surface, such as from smiling or frowning muscles, the invention relies on capillary adhesion and friction between the smooth intraoral device surface and the buccal mucosa. A friction coefficient of approximately 0.2 is used, such that resisting a 2-newton tangential force requires a normal adhesive force of approximately 10 newtons. Capillary pressure may reach approximately 133,000 pascals in a central zone under favorable thin-film conditions, but an adjusted average capillary pressure of approximately 50,000 pascals is more realistic when accounting for curvature, tissue compliance, and uneven fluid distribution. Under the realistic average, the invention specifies an effective adhesive contact area of approximately 2.0 square centimeters in the central region to generate the required adhesive normal force. Nighttime conditions are explicitly addressed because saliva production decreases significantly during sleep, affecting adhesion. Under nighttime conditions, the central saliva film thickness is approximately 1.5 micrometers, while saliva films toward the edges may thicken up to approximately 3-5 micrometers due to fluid pooling and reduced muscle movement. The thickness variation reduces effective capillary pressure particularly at the periphery, creating “edge effects” that reduce the fraction of the total area contributing meaningful adhesion. The invention therefore specifies that only about 45% of the total lobe contact area may contribute effective capillary adhesion under nighttime conditions, and accordingly increases the total required contact area to approximately 4.44 square centimeters to ensure stable retention. For geometric intuition, if the total contact area were circular, it would correspond to a diameter of approximately 2.38 centimeters. These quantitative parameters support design of lobe geometry and surface area to maintain inhibition facial muscle contractions and device stability under nocturnal muscle movements' tangential forces.

The present invention emphasizes that suctional contact areas are not merely incidental, but constitute a primary retention mechanism that works in conjunction with physical engagement to the dentition. The dual-retention approach reduces the need for excessive bulk, aggressive clamping, or overextension, thereby enhancing comfort, wearability, and stability across different users and operating conditions. The suctional interface stabilizes the device against lateral sliding and micro-displacement caused by facial muscle activity, while the dentition interface provides gross positional anchoring. A further design constraint is minimizing overstretching of surrounding soft tissues. The invention expressly targets sufficient surface suction force for inhibition and stability without overfilling available free intraoral space. Overfilling can cause excessive stretching of buccal soft tissue and may lead to strain or dysfunction of the buccinator (trumpeter's) muscle. Accordingly, the device geometry, material compliance, lobe span, and contact area are engineered to provide adequate adhesion, inhibition and support while maintaining physiologically safe soft-tissue deformation and preserving normal muscle function.

In use, the intraoral apparatus may be worn for extended durations, including overnight wear of approximately 6-8 hours daily, to promote long-term prevention and reduction of nasolabial folds by stabilizing facial soft tissues during resting states and reducing unintended facial muscle activation during sleep. The invention is further suitable for maintenance of facial appearance following injectable filler treatment, where sustained intraoral support may reduce mechanical stresses that contribute to recurrence or worsening of nasolabial folds. The invention thereby provides a technically engineered, non-invasive intraoral system integrating anatomical positioning, material properties, surface physics, and biomechanical retention principles to achieve stable intraoral wear and effective long-term management of nasolabial folds.

The intraoral apparatus described herein provides numerous technical and functional advantages over conventional approaches for managing nasolabial folds. The apparatus operates entirely within the oral cavity and therefore avoids surgical procedures, injectable treatments, external facial devices, and associated risks such as scarring, bruising, swelling, infection, or downtime. By combining physical engagement with the dentition and suction-based capillary adhesion at smooth lobe surfaces, the apparatus achieves inhibition of facial muscle activation and stable intraoral retention without excessive bulk or aggressive clamping, thereby improving comfort and wearability during extended use, including overnight wear. The anatomically defined lobe positioning and controlled extension below the occlusal plane enable targeted support of facial soft tissues underlying nasolabial folds while minimizing overstretching of surrounding tissues and avoiding strain or dysfunction of facial muscles such as the buccinator muscle. The use of medical-grade elastomeric materials with defined durometer ranges and glassy-smooth surface finishes enhances conformity with buccal mucosa, promotes consistent suction under varying saliva conditions, inhibition of muscle activation, and reduces slippage caused by facial muscle movement. The apparatus may be provided in custom-fit or standardized configurations, with optional reinforcement elements for adjustability, allowing broad applicability across different users and anatomical variations. Through repeated, prolonged use, the apparatus supports facial tissues during resting states, reduces repetitive tissue deformation, and may prevent formation, slow progression, reduce appearance, or maintain cosmetic improvements of nasolabial folds in a cost-effective, non-invasive, and user-controlled manner.

The foregoing descriptions of specific embodiments of the present technology have been presented for purposes of illustration and description. They are not intended to be exhaustive or to limit the present technology to the precise forms disclosed, and obviously many modifications and variations are possible considering the above teaching. The embodiments were chosen and described to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology. While several possible embodiments of the invention have been described above and illustrated in some cases, it should be interpreted and understood as to have been presented only by way of illustration and example, but not by limitation. Thus, the breadth and scope of a preferred embodiment should not be limited by any of the above-described exemplary embodiments.

What is claimed is:

1. An intraoral apparatus for preventing or reducing nasolabial folds, comprising:
an intraoral support structure configured to be positioned within an upper oral cavity of a user;
an upstanding wall configured to extend along buccal surfaces of upper teeth, the upstanding wall having a top portion oriented toward an upper lip and a bottom portion oriented toward a gingival region;
two lobes positioned apart on opposing lateral sides of the upstanding wall and extending outwardly from the upstanding wall, each lobe being configured to engage inner cheek tissue in regions underlying nasolabial folds;
wherein the lobes have a greater thickness than adjacent portions of the upstanding wall and define outwardly bulged tissue-contacting surfaces, and
wherein the apparatus is configured to be retained intraorally by a combination of engagement with the upper teeth and surface adhesion between the lobes and buccal mucosa.

2. The intraoral apparatus of claim 1, wherein the apparatus further comprises a tray configured to cover occlusal surfaces of the upper teeth, the tray cooperating with the upstanding wall to stabilize the apparatus within the upper jaw.

3. The intraoral apparatus of claim 2, wherein the tray and the upstanding wall define a tooth-receiving surface having recesses configured to receive and conform to the upper teeth.

4. The intraoral apparatus of claim 1, wherein the upstanding wall includes a smooth inner surface configured to contact buccal surfaces of the upper teeth without individual tooth recesses.

5. The intraoral apparatus of claim 1, wherein each lobe extends upwardly toward a junction of the upper lip and gingiva and extends downwardly below an occlusal plane of the upper teeth.

6. The intraoral apparatus of claim 1, wherein each lobe spans longitudinally from a mesial region of a first premolar to a distal region of a first molar.

7. The intraoral apparatus of claim 1, wherein each lobe extends downwardly below an occlusal plane by approximately five millimeters to 1 cm and spans from a mesial surface of a first premolar to approximately a midline of a second molar.

8. The intraoral apparatus of claim 1, wherein the lobes are formed from a medical-grade elastomeric material having a Shore A hardness in a range of approximately 20 to 60.

9. The intraoral apparatus of claim 1, wherein the lobes have a glassy-smooth surface finish configured to promote formation of a saliva film between the lobes and buccal mucosa to generate capillary adhesion.

10. The intraoral apparatus of claim 1, wherein the lobes define a total tissue-contacting surface area of approximately 4.44 square centimeters to resist tangential forces generated by facial muscle movement during use.

11. The intraoral apparatus of claim 1, further comprising a handle coupled to the upstanding wall and configured to facilitate insertion, alignment, or removal of the apparatus from the oral cavity.

12. The intraoral apparatus of claim 1, further comprising a bendable reinforcement element positioned within the upstanding wall and configured to permit manual adjustment of curvature or orientation of the upstanding wall and the lobes.
